(19) United States
(12) Patent Application Publication
(10) Pub. No.: US 2005/0277846 A1
(43) Pub. Date: Dec. 15, 2005

(54) Cervical tissue sampling device

(76) Inventor: Chun-Shan Chou, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/863,465

(22) Filed: Jun. 9, 2004

(51) Int. Cl. A61B 10/00

(52) U.S. Cl. 600/569; 600/562

(57) ABSTRACT

A cervical tissue sampling device includes an exterior tube, a hold and an interior shaft. The exterior tube has an opening formed on a tapered end thereof and an internal screw thread arranged therein. The hold includes an operation base and a propelling plate connecting the operation base upwardly, wherein the propelling plate is sleeved on by the exterior tube. The interior shaft is adjacent to the propelling plate, arranged above the operation base and received in the exterior tube. The interior shaft includes a projective screw thread portion disposed on an end thereof, and a fluff portion arranged on an opposite end thereof. The projective screw thread portion abuts against and is pushed by the propelling plate to move upwards due to a match for the projective screw thread portion and the internal screw thread, so as to expose the fluff portion outside the exterior tube via the opening.

### BACKGROUND OF THE INVENTION

1. Field of the Invention

The present invention relates to a cervical tissue sampling device, and particularly relates to a cervical tissue sampling device adopted for a Pap smear and easy for manipulation without obvious feeling of foreign body.

2. Background of the Invention

The cervix is the opening between the vagina and the uterus. It is located at the top of the vagina. When cervical cancer, a common cancer in women, develops, the cells of the cervix become abnormal and grow out of control, and the cancer can spread to other parts of the body. A Pap smear can find the cervical cancer before it moves to other parts of your body and becomes invasive. If caught early, treatment for the cervical cancer can be easier and the chances of curing it are far greater. The Pap smear can also pick up infections and inflammation, vaginitis, abnormal cells that can change into cancer cells, and some sexually transmitted diseases.

The Pap smear is usually done by a practiced nurse, or at a family planning clinic. However, because of shy or fear of lying on the table, almost half of women have never had the smear and 15% of them haven't had one for five years, who are newly diagnosed with the cervical cancer. For ease to implement the Pap smear, a simple and convenient device, like the Pap smear, that can help women take tissue samples from the cervix by themselves with privacy is provided. FIG. 1 illustrates a conventional cervical tissue sampling device 1a, which includes an interior shaft 2a, an exterior tube 3a enclosing the interior shaft 2a, and a sampling bell 4a disposed an end of the exterior tube 3a. The interior shaft 2a includes a fluff head 21a arranged thereof and surrounding by the sampling bell 4a, and the sampling bell 4a includes an inner fluff wall 41a arranged thereof. When the conventional cervical tissue sampling device 1a is put into the vagina to attempt to scrape some cells from the surface and the inside of the cervix, the sampling bell 4a can be spread and stretched outwardly to expose the inner fluff wall 41a. The inner fluff wall 41a and the fluff head 21a can touch the surface of the cervix simultaneously to take tissue samples. Although the inner fluff wall 41a is soft and resilient to be retracted back and extended alternatively, the configuration thereof is still lager than that of the exterior tube 3a, and that causes feeling of foreign body obviously.

Hence, an improvement over the prior art is required to overcome the disadvantages thereof.

### SUMMARY OF INVENTION

The primary object of the invention is therefore to specify a cervical tissue sampling device, as a Pap smear, that provides easy manipulation for women doing it by themselves, with privacy but without obvious feeling of foreign body.

According to the invention, the object is achieved by a cervical tissue sampling device including an exterior tube, a hold and an interior shaft. The exterior tube has an opening formed on a tapered end thereof and an internal screw thread arranged therein. The hold includes an operation base and a propelling plate connecting the operation base upwardly. The propelling plate is sleeved on by the exterior tube. The interior shaft is adjacent to the propelling plate, arranged above the operation base and received in the exterior tube. The interior shaft includes a projective screw thread portion disposed on an end thereof, and a fluff portion arranged on an opposite end thereof. The projective screw thread portion abuts against and is pushed by the propelling plate to move upwards due to a match for the projective screw thread portion and the internal screw thread, so as to expose the fluff portion outside the exterior tube via the opening.

To provide a further understanding of the invention, the following detailed description illustrates embodiments and examples of the invention. Examples of the more important features of the invention thus have been summarized rather broadly in order that the detailed description thereof that follows may be better understood, and in order that the contributions to the art may be appreciated. There are, of course, additional features of the invention that will be described hereinafter and which will form the subject of the claims appended hereto.

### BRIEF DESCRIPTION OF THE DRAWINGS

These and other features, aspects, and advantages of the present invention will become better understood with regard to the following description, appended claims, and accompanying drawings, where:

FIG. 1 is a cross-sectional profile of a conventional cervical tissue sampling device;

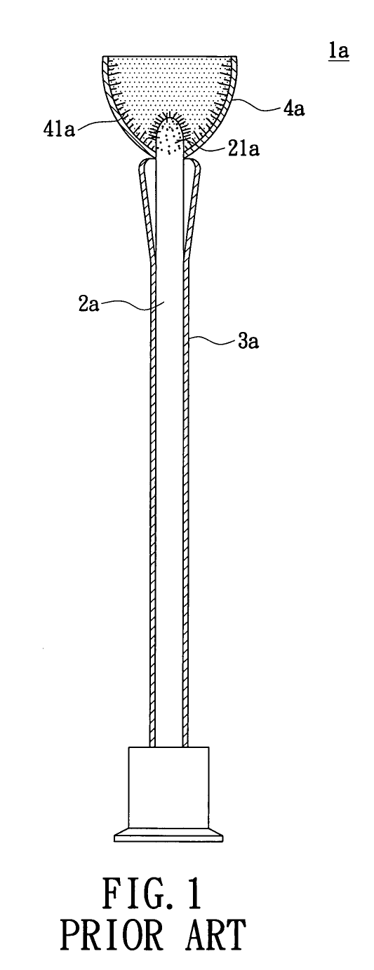

FIG. 2 is a perspective view of a cervical tissue sampling device according to a preferred embodiment of the present invention;

FIG. 3 is an explosion view of the cervical tissue sampling device according to the preferred embodiment the present invention;

FIG. 4 is a cross-sectional profile of the cervical tissue sampling device according to the preferred embodiment of the present invention;

FIG. 5 is a perspective view of another embodiment of the cervical tissue sampling device according to the present invention;

FIG. 6 is a perspective view of an application of the cervical tissue sampling device before a cell-scrapping process according to the present invention; and

FIG. 7 is a perspective view of the application of the cervical tissue sampling device during a cell-scrapping process according to the present invention.

### DETAILED DESCRIPTION OF THE EMBODIMENTS

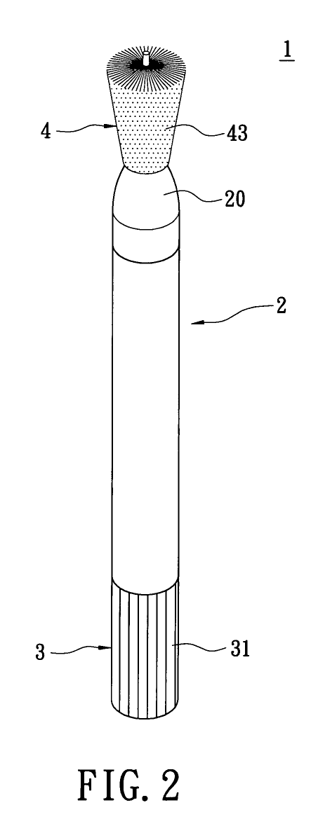

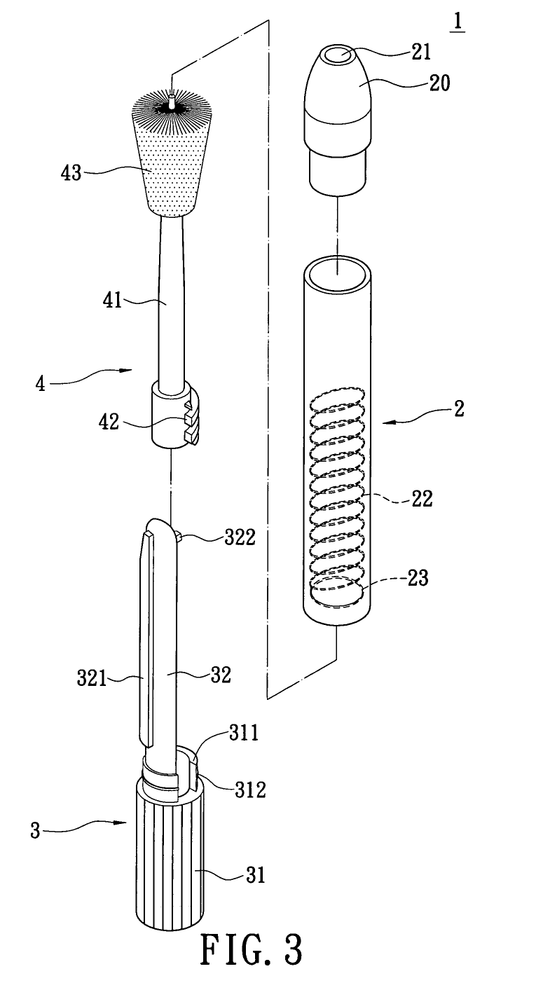

With respect to FIGS. 2 and 3, the present invention provides a cervical tissue sampling device 1 includes an exterior tube 2, a hold 3, an interior shaft 4, and a stop means. The exterior tube 2 has a tube body, an opening 21 formed on a tapered end 20 connecting the tube body, and an internal screw thread 22 arranged in the tube body. The tapered end 20 of the exterior tube 2 has a smooth surface due to a gradually narrow diameter; therefore, the cervical tissue sampling device 1 will not make obvious feeling of foreign body, and that can raise the will of the exam. The tapered end 20 can be connected to the tube body in a detachable manner; alternatively, the tapered end 20 can be integrally extended from the tube body in one piece. In this embodiment, the tapered end 20 is connected to the tube body in a detachable manner.

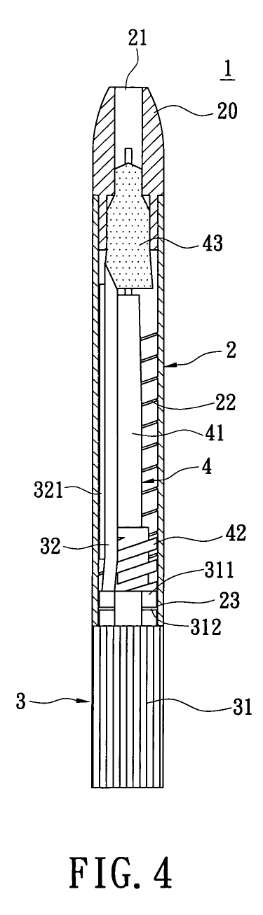

The hold 3 includes an operation base 31 and a propelling plate 32 connecting the operation base 31 upwardly. The propelling plate 32 is sleeved on by the exterior tube 2 to mate with the operation base 31. The operation base 31 has a pattern projecting from a surface thereof to provide an easy grab. The interior shaft 4 is adjacent to the propelling plate 32, arranged above the operation base 32 and received in the exterior tube 2. The interior shaft 4 includes a shaft body 41, a projective screw thread portion 42 disposed on a lower end of the shaft body 41, and a fluff portion 43 arranged on an opposite end thereof. The fluff portion 43, as the conventional cervical tissue sampling device 1a presents, is provided for scrapping tissue samples. The projective screw thread portion 42 protrudes to abut against the propelling plate 32 and mate with the internal screw thread 22 simultaneously. Referring to FIG. 4, if the propelling plate 32 is rotated by the operation base 31, the projective screw thread portion 42 is pushed by the propelling plate 32 to move upwards due to the match between the projective screw thread portion 42 and the internal screw thread 22, so as to expose the fluff portion 43 outside the exterior tube 2 via the opening 21.

In addition, the hold 3 has a pair of resilient shells 311 extending upwardly from the operation base 31 for accommodation with the exterior tube 2. The exterior tube 2 includes an internal circular ring 23 extending inwardly from a bottom thereof, and each of the resilient shells 311 has an annular projection 312 arranged therefrom to engage with the internal circular ring 23 of the exterior tube 2.

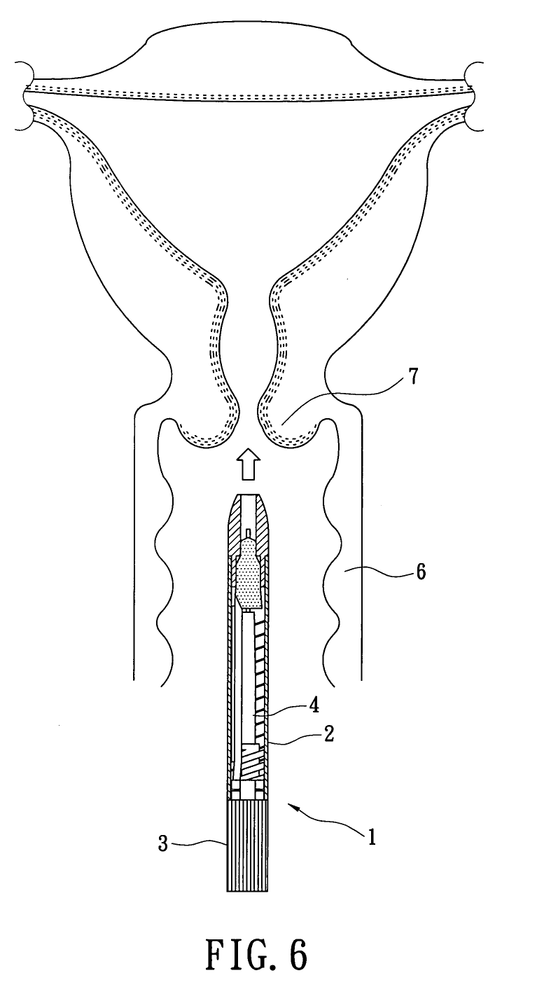

The propelling plate 32 has a withstanding portion 321 (shown in FIG. 3) disposed thereon and abutting against the exterior tube 22, in order to oppress the projective screw thread portion 42 of the interior shaft 4 to touch the exterior tube 22 tightly. The propelling plate 32 extends from the operation base 31 upwardly with a predetermined length, which indicates a moving length of the interior shaft 4. Thus, the cervical tissue sampling device 1 according to the present invention can extend the interior shaft 4 to a certain depth in the vagina 6 (see FIG. 6).

The stop means is used for avoiding the interior shaft 4 departing from the exterior tube 2. In the preferred embodiment, the stop means can be a terminated end, not a free-running end, arranged on a top of the internal screw thread 22, so as to discontinue an upward moving of the interior shaft 4. Alternatively, the stop means can be a protrusion 322 (shown in FIG. 2) extending a top of the propelling plate 322, so as to discontinue an upward moving of the interior shaft 4, too.

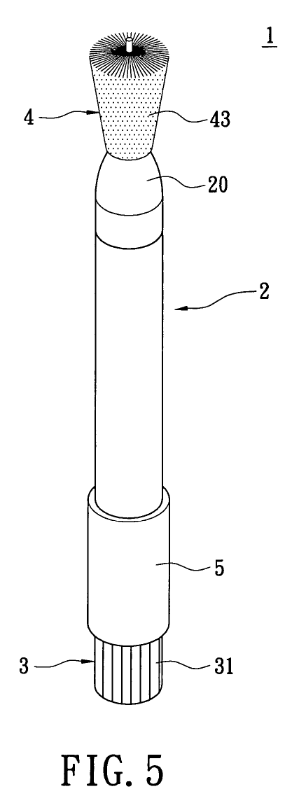

For ease to manipulating the cervical tissue sampling device 1, FIG. 5 illustrates a slip-proof sleeve 5 sleeved on the cervical tissue sampling device 1. The slip-proof sleeve 5 is resilient for touching with comfortable. The slip-proof sleeve 5 can be arranged to sleeve on the exterior tube 2 and the hold 3 simultaneously.

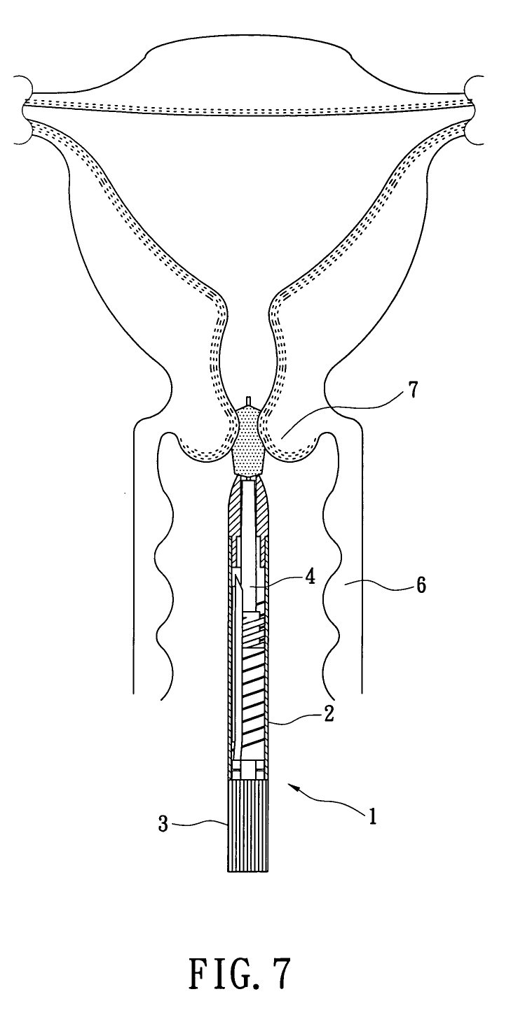

With respect to FIGS. 6 and 7, the cervical tissue sampling device 1 is inserted into the vagina 6 first with beforehand retraction of the interior shaft 4, the operation base 31 is rotated to carry the interior shaft 4 to move upwardly. Women can clip the slip-proof sleeve 5 that may be applicable in FIGS. 6 and 7, while the operation base 31 is rotated. Until the fluff portion 43 is forced to expose outside the exterior tube 2 and touch the cervix 7. The cervical tissue sampling device 1 can be further turn right or left to scrap more cells and tissues. After the cells and tissues are taken, the operation base 31 is rotate reversely to retract the interior shaft 4 back. To take the cervical tissue sampling device 1 out of the vagina 6, the tissue sample taken process is achieved thereby.

The present invention is characterized by:
1. The present invention provides a privacy exam without obvious feeling of foreign body.
2. The present invention provides a convenient manipulation for women doing it by themselves.

It should be apparent to those skilled in the art that the above description is only illustrative of specific embodiments and examples of the invention. The invention should therefore cover various modifications and variations made to the herein-described structure and operations of the invention, provided they fall within the scope of the invention as defined in the following appended claims.

1. A cervical tissue sampling device, comprising:
an exterior tube having an opening formed on a tapered end thereof and an internal screw thread arranged therein;
a hold including an operation base and a propelling plate connecting the operation base upwardly, wherein the propelling plate is sleeved on by the exterior tube; and
an interior shaft adjacent to the propelling plate, arranged above the operation base and received in the exterior tube, wherein the interior shaft includes a projective screw thread portion disposed on an end thereof, and a fluff portion arranged on an opposite end thereof;
whereby the projective screw thread portion abuts against and is pushed by the propelling plate to move upwards due to a match for the projective screw thread portion and the internal screw thread, so as to expose the fluff portion outside the exterior tube via the opening.

2. The cervical tissue sampling device as claimed in claim 1, wherein the hold has a pair of resilient shells extending upwardly from the operation base for accommodation with the exterior tube.

3. The cervical tissue sampling device as claimed in claim 2, wherein the exterior tube includes an internal circular ring extending inwardly from a bottom thereof, and each of the resilient shells has an annular projection arranged therefrom to engage with the internal circular ring of the exterior tube.

4. The cervical tissue sampling device as claimed in claim 1, further including a stop means for avoiding the interior shaft departing from the exterior tube.

5. The cervical tissue sampling device as claimed in claim 4, wherein the stop means is a terminated end arranged on a top of the internal screw thread, so as to discontinue an upward moving of the interior shaft.

6. The cervical tissue sampling device as claimed in claim 4, wherein the stop means is a protrusion extending a top of the propelling plate, so as to discontinue an upward moving of the interior shaft.

7. The cervical tissue sampling device as claimed in claim 1, wherein the propelling plate has a withstanding portion disposed thereon and abutting against the exterior tube, in order to oppress the projective screw thread portion of the interior shaft to touch the exterior tube tightly.

8. The cervical tissue sampling device as claimed in claim 1, wherein the propelling plate extends from the operation base upwardly with a predetermined length, which indicates a moving length of the interior shaft.

9. The cervical tissue sampling device as claimed in claim 1, wherein the operation base has a pattern projecting from a surface thereof.

10. The cervical tissue sampling device as claimed in claim 1, further including a slip-proof sleeve sleeved thereon, the slip-proof sleeve being resilient.
